(19) United States
(12) Patent Application Publication
(10) Pub. No.: US 2006/0241760 A1
(43) Pub. Date: Oct. 26, 2006

(54) Spinal implant

(76) Inventors: Brandon Randall, West Chester, PA (US); Kenneth Kobayashi, Exton, PA (US); Dominique Messerli, Downington, PA (US)

Correspondence Address: JONES DAY, 222 EAST 41ST ST, NEW YORK, NY, 10017, US

(21) Appl. No.: 11/114,556

(22) Filed: Apr. 26, 2005

(51) Int. Cl. A61F 2/44

(52) U.S. Cl. 623/17.11

(57) ABSTRACT

The present invention is directed to a spinal implant, preferably a bioresorbable spinal implant, for insertion between vertebrae bodies. The implant preferably includes an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface. The central bore preferably has a generally lobe-shaped footprint. The anterior surface of the implant preferably includes a pair of vertical channels sized and configured to engage an insertion instrument and the superior surface preferably has a convexly curved surface extending substantially from the anterior surface to the posterior surface while the inferior surface preferably has a substantially constant taper extending from the anterior surface to the posterior surface.

### FIELD OF THE INVENTION

The present invention relates to a spinal implant used in spinal fusion procedures. More specifically, the present invention relates to a bioresorbable implant for use in spinal fusion to replace an intervertebral disk and/or a vertebral body in combination with its corresponding intervertebral disks.

### BACKGROUND OF THE INVENTION

A number of medical conditions such as compression of spinal cord nerve roots, degenerative disk disease, and trauma can cause severe back pain. Intervertebral fusion is a surgical method of alleviating back pain. In intervertebral fusion, two adjacent vertebral bodies are fused together by removing the affected intervertebral disk and inserting an implant that would allow for bone to grow between the two vertebral bodies to bridge the gap left by the disk removal. A corpectomy is a surgical procedure wherein a vertebral body is removed in combination with its associated intervertebral disk(s). In a corpectomy, two vertebral bodies are fused together by removing the affected vertebral body in combination with its associated intervertebral disks and inserting an implant that would allow for bone to grow between the two vertebral bodies to bridge the gap left by the vertebral and disk removal.

A number of different implants and implant materials have been used for fusion with varying success. For example, current implants are manufactured from stainless steel, titanium, titanium alloy, allografts, a metal-allograft composite, polymers, plastics, ceramics, etc. Titanium cages suffer from a number of disadvantages. For example, due to MRI incompatibility of titanium, determining fusion is problematic. Furthermore, restoration of lordosis, i.e., the natural curvature of the cervical and lumbar spine is very difficult when a titanium cage is used.

Allografts are sections of bone usually taken from the diaphysis of a long bone, such as the radius, ulna, fibula, humerus, tibia, or femur of a donor. A cross section of the bone is taken and processed using known techniques to preserve the allograft until implantation and reduce the risk of an adverse immunological response when implanted. Allografts have mechanical properties which are similar to the mechanical properties of vertebrae even after processing. This prevents stress shielding that occurs with metallic implants. They are also MRI compatible so that fusion can be more accurately ascertained and promote the formation of bone, i.e., osteoconductive. Although the osteoconductive nature of the allograft provides a biological interlocking between the allograft and the vertebrae for long term mechanical strength, initial and short term mechanical strength of the interface between the allograft and the vertebrae is generally lacking such that there is a possibility of the allograft being expelled after implantation.

Furthermore, most allografts are simple sections of bone which, although cut to the approximate height of the disk being replaced, have not been sized and/or machined on the exterior surface to have a uniform shape. As a result, the fusion of the vertebral bodies does not occur in optimal anatomic position in a consistent manner along the surface of the endplates. While a surgeon may do some minimal intraoperative shaping and sizing to customize the allograft for the patient's anatomy, significant shaping and sizing of the allograft is not possible due to the nature of the allograft. Even if extensive shaping and sizing were possible, a surgeon's ability to manually shape and size the allograft to the desired dimensions is severely limited.

Moreover, with the rapidly increasing demand in the medical profession for devices incorporating allograft material, the tremendous need for allograft material itself, presents a considerable challenge to the industry that supplies the material.

As the discussion above illustrates, there is a need for a spinal implant whose design takes into consideration the anatomy and geometry of the space sought to be filled by the implant as well as the anatomy and geometry of the end plates of the vertebral bodies. There is also a need for a spinal implant which can be readily visualized due to its radiopaque properties and one which integrates well with the vertebral bone tissue of the vertebral bodies between which the implant is to be inserted.

### SUMMARY OF THE INVENTION

The present invention relates to a spinal implant for insertion between vertebrae bodies, the implant having an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein the central bore has a generally lobe-shaped footprint, which includes a plurality of peeks and valleys as one moves along a perimeter of the bore.

The present invention further relates to a spinal implant for insertion between vertebrae bodies wherein the implant has an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein the anterior surface of the implant includes a pair of vertical channels sized and configured to engage an insertion instrument.

The present invention further relates to a spinal implant for insertion between vertebrae bodies wherein the implant has an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein one of the superior and inferior surfaces has a convexly curved surface extending substantially from the anterior surface to the posterior surface and wherein the other one of the superior and inferior surfaces has a substantially constant taper extending from the anterior surface to the posterior surface, the superior and inferior surfaces both having a convexly curved surface extending from one lateral surface to the other lateral surface.

In an alternate embodiment of the present invention, preferably, at least a portion of the anterior and posterior surfaces are curved and the lateral surfaces are substantially convex for mating with the curved anterior and posterior surfaces. Moreover, the posterior surface may include a concave recess for avoiding the foramen of the vertebral bodies when inserted. The superior and inferior surfaces may include a plurality of gripping structures, preferably teeth, formed thereon to facilitate engagement of the implant with the vertebrae bodies.

Furthermore, the implant preferably has a generally wedge-shaped profile extending from the anterior surface to the posterior surface. More preferably, one of the superior and inferior surfaces has a convexly curved surface extending substantially from the anterior surface to the posterior surface while the other one of the superior and inferior surfaces has a substantially constant taper extending from the anterior surface to the posterior surface. The superior and inferior surfaces preferably both have a convexly curved surface extending from one lateral surface to the other lateral surface.

The implant also preferably includes a pair of vertical channels sized and configured to engage an insertion instrument such that the insertion instrument is sized and configured to engage the implant so that, once engaged, the insertion instrument has a width substantially equal to the width of the implant.

The implant also preferably has at least one radiopaque marker embedded within the implant. The radiopaque marker may be a tooth filled with radiopaque material. Alternatively, the radiopaque marker may involve coating at least a portion of an outer surface of the central bore with radiopaque material. The radiopaque marker may involve placing a reference marker on one of the surfaces of the implant, the reference marker being made from a radiopaque material. The radiopaque marker may involve filling at least one void formed in the implant with radiopaque material. The radiopaque marker may involve marking at least a portion of the outer perimeter of the implant with a radiopaque stripe.

The central bore of the implant preferably is filled a bone growth inducing substance. Moreover, the implant may include at least one void in a surface thereof which is also filled with the bone growth inducing substance.

The implant is preferably formed of a bioresorbable material.

### BRIEF DESCRIPTION OF THE DRAWINGS

FIG. 1 is a top view of an embodiment of the spinal implant according to the present invention;

FIG. 2 is a cross-sectional side view of the spinal implant shown in FIG. 1;

FIG. 3 is an anterior view of the spinal implant shown in FIG. 1;

FIG. 4 is a detailed view of the vertical channel formed in the implant;

FIG. 5 is an embodiment of a radiopaque marker used in conjunction with the implant shown in FIG. 1;

FIG. 6 is another embodiment of a radiopaque marker used in conjunction with the implant shown in FIG. 1;

FIG. 7 is another embodiment of a radiopaque marker used in conjunction with the implant shown in FIG. 1;

FIG. 8 is another embodiment of a radiopaque marker used in conjunction with the implant shown in FIG. 1;

FIG. 9 is another embodiment of a radiopaque marker used in conjunction with the implant shown in FIG. 1;

FIG. 10 is another embodiment of a radiopaque marker used in conjunction with the implant shown in FIG. 1;

FIG. 11 is an embodiment of the implant shown in FIG. 1 incorporating at least one void for receiving bone growth inducing substances.

### DETAILED DESCRIPTION OF THE PREFERRED EMBODIMENTS

For the purposes of promoting an understanding of the principles of the present invention, reference will now be made to an exemplary, non-limiting embodiment illustrated in the drawings and specific language will be used to describe the same. It will nevertheless be understood that no limitation of the scope of the invention is hereby intended, such alterations and further modifications in the illustrated devices, and such further applications of the principles of the invention as illustrated therein being contemplated as would normally occur to one of ordinary skill in the art to which the invention relates.

The present invention is directed a spinal implant 10 for insertion between vertebral bodies. The implant 10 is preferably sized and configured to restore the height of the removed disk space, to provide immediate load bearing capability and to support the vertebral column. Although the implant 10 can be formed from any biological material known to one of ordinary skill in the art such as, for example, stainless steel, titanium, titanium alloy, allograft, a metal-allograft composite, polymers, plastics, ceramics, etc. Preferably, the implant 10 is formed from a bioresorbable material such as, for example, poly-L-lactide, poly-D-lactide, polyglycolide, polycarbonate (from Rutgers University), and combinations thereof. More preferably, the implant 10 is formed from a bioresorbable material and includes at least one radiopaque marker embedded within the implant 10 to aide in visualization of the implant, as will be described in greater detail below.

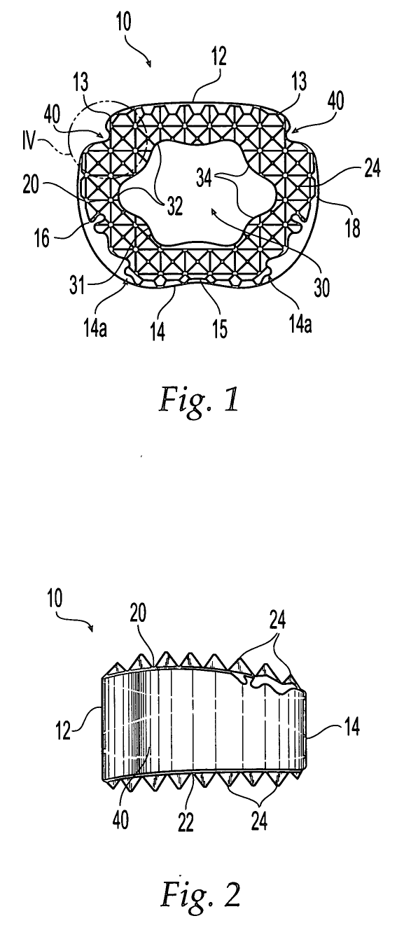

As shown in FIG. 1, the spinal implant 10 has a generally kidney bean footprint and includes an anterior surface 12, a posterior surface 14, and first and second lateral surfaces 16, 18 extending therebetween. As shown, the anterior surface 12 is substantially straight with a pair of arcuate end portions 13 while the posterior surface 14 has a pair of arcuate end portions 14a and a concave recess 15. The lateral surfaces 16, 18 interconnect the anterior and posterior surfaces 12, 14 and are substantially arcuate, preferably convex, in shape. More preferably, the arcuate end portions 13 of the anterior surface 12 are convex and have a radius of curvature between about 3 mm and 10 mm, more preferably about 5.5 mm. The arcuate end portions 14a of the posterior surface 14 have a radius of curvature between about 5 mm and 10 mm, more preferably 7.5 mm, while the concave recess 15 has a radius of curvature between about 5 mm and 200 mm, more preferably about 7.5 mm.

In use, the footprint of the spinal implant 10 is preferably designed to conform in size and shape with the general perimeter and shape of the end plates of the vertebrae between which the implant 10 is inserted thereby providing maximum support. To this end, the incorporation of the recess 15 in the posterior surface 14 of the implant 10 is sized and configured to avoid the intravertebral foramen of the vertebral bodies. The intravertebral foramen or the spinal canal is the portion of the vertebral body that houses the spinal cord and nerve roots. Generally, a portion of the intravertebral foramen extends into the body portion or end plate portion of the vertebra. This portion of the intravertebral foramen, in effect, changes the perimeter of the body portion of the vertebra from substantially an oval shape to substantially a kidney-bean shape. Accordingly, the footprint of the spinal implant 10 is generally kidney-bean shaped to emulate the general shape and perimeter of the body portion of the vertebrae.

The spinal implant 10 also has a superior surface 20 and an inferior surface 22 which incorporate a plurality of gripping structures 24 formed thereon to facilitate engagement of the implant 10 with the end plates of the vertebra. That is, the gripping structures 24 provide a mechanical interlock between the implant 10 and the end plates of the vertebrae by penetrating the end plates. This initial mechanical stability, afforded by the gripping structures 24 minimizes the risk of post-operative expulsion or slippage of the implant 10. Although any gripping structure 24 known in the art may be used, for example, undulating surfaces, projections, saw-tooth teeth, ridges, etc. Preferably, the superior and inferior surfaces 20, 22 include a plurality of pyramidal teeth. Alternatively or in addition, the implant 10 may include areas extending from an outer periphery of the implant 10 which are essentially smooth and devoid of any gripping structure 24 for receiving a surgical instrument. The substantially smooth areas may extend in an anterior-posterior direction, a lateral direction, or may run in both directions. In addition, the substantially smooth area may run in an anterio-lateral direction. The superior and inferior surfaces 20, 22 may also include a beveled edge, as will be described in greater detail below.

The spinal implant 10 may also include a central bore 30 extending from the superior surface 20 to the inferior surface 22, which can be filled with bone growth inducing substances to allow for bony in-growth, as will be described in greater detail below, and to further assist in the fusion of the vertebrae and the implant 10. As best shown in FIG. 1, the central bore 30 has a generally lobe-shaped footprint, which includes a plurality of peeks 32 and valleys 34 as one moves along the perimeter 31 of the bore 30. In this regard, the lobe-shape central bore 30 increases the surface area of the implant 10 in contact with the endplates of the vertebrae. Thus, where the implant 10 is formed from a bioresorbable material, the lobe-shaped central bore 30 helps facilitate absorption of the implant 10. Preferably, the plurality of peeks 32 and valleys 34 forming the lobe-shaped outer perimeter 31 of the central bore 30 have a radius of curvature between about 1 mm and 4 mm, more preferably about 2 mm. Alternatively, the peeks 32 and valleys 34 may end in a sharp point (not shown).

Referring to FIG. 2, the implant 10 preferably has a generally wedge-shaped profile extending from the anterior surface 12 to the posterior surface 14 so that the implant 10 helps restore the natural curvature or lordosis of the spine after the affected disk has been removed. More specifically, unlike prior art implants which generally have tapering superior and inferior surfaces or convex superior and inferior surfaces, the superior surface 20 of the implant 10 preferably has a curved, more preferably convex, surface from the anterior surface 12 to the posterior surface 14 while the inferior surface 22 preferably has a constant taper extending from the anterior surface 12 to the posterior surface 14. This configuration ensures that the implant 10 most appropriately matches the anatomy of the spine, especially when the implant 10 is used in the cervical region of the spine. As a result of this configuration, the implant 10 has a gradually increasing height extending from the anterior surface 12 followed by a gradually decreasing height as the posterior surface 14 is approached so that the height of the implant 10 at the posterior surface 14 is less than the height of the implant 10 at the anterior surface 12 with the greatest height occurring someplace therebetween. Preferably, the convex superior surface 20 has a radius of curvature between about 50 mm and 200 mm—more preferably about 100 mm—for lumbar applications, a radius of curvature between about 25 mm and 150 mm—more preferably about 65 mm—for thoracic applications, and a radius of curvature between about 5 mm and 25 mm—more preferably about 14 mm—for cervical applications. Preferably, the tapered inferior surface 22 tapers at an angle of about 3.5 degrees. Alternatively, the superior surface 20 may have the constant taper while the inferior surface 22 may have the curved, preferably convex, surface. However, it should be noted that the superior and inferior surfaces 20, 22 may have matching convex and/or tapering surfaces.

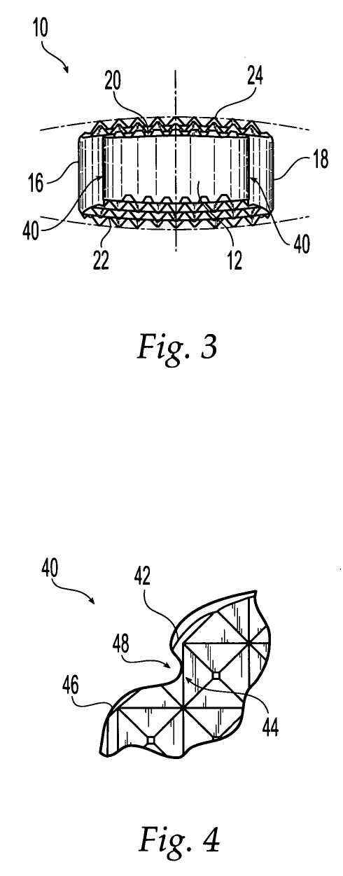

Referring to FIG. 3, the superior and inferior surfaces 20, 22 of the implant 10 are curved, preferably convex, when viewed from the anterior and/or posterior surfaces so that the thickness of the implant 10 is greatest at the mid-section between the two lateral side surfaces 16, 18 and tapers gradually along the lateral axis of the implant 10 so that the height of the implant 10 is thinnest at the lateral side surfaces 16, 18. This convex configuration helps provides a proper anatomical fit. The height of the implant 10 at the lateral ends 16, 18 may or may not be the same. Preferably, the convex surfaces have a radius of curvature of about 50 mm.

In order to facilitate insertion of the implant 10, the anterior surface 12 preferably includes a pair of vertical channels 40, as best shown in FIGS. 1 and 4, which may extend from the superior surface 20 to the inferior surface 22. The vertical channels 40 are sized and configured to mate with a releasably engaging insertion instrument (not shown) to enable a surgeon to fixedly engage the implant 10 during the insertion procedure. More specifically, the channels 40 are sized and configured to engage projections formed on the insertion instrument. As best shown in FIG. 4, the vertical channels 40 preferably have a compound curved surface formed by arcuate curves 42, 44, and 46 wherein arcuate curve 42 forms the transition between the anterior surface 12 and the vertical channel 40. Arcuate surface 46 forms the transition between the lateral surface and the vertical channel 40 and arcuate surface 44 substantially interconnects arcuate surface 42 and 46. In this configuration, the vertical channel 40 provides a bearing surface 48 against which the projections formed on the releasably engaging insertion instrument may contact. More preferably, arcuate surface 42 has a radius of curvature of about 0.4 mm, arcuate surface 44 has a radius of curvature of about 0.5 mm and arcuate surface 46 has a radius of curvature of about 0.5 mm.

As shown, the channels 40 are preferably located on the anterior surface 12 of the implant 10 such that the insertion instrument, when engaged, does not extend laterally beyond the lateral surfaces 16, 18 of the implant 10. Moreover, preferably, the insertion instrument is sized and configured so that its height, when engaged, is less than or equal to the height of the implant 10. This configuration helps minimize the trauma associated with the insertion of the implant 10 by ensuring that the insertion tool does not project beyond the footprint of the implant 10. To this end preferably, the vertical channels 40 are located approximately 34 degrees from the lateral axis of the implant 10. Although the channels 40 are shown as extending the entire height of the implant 10, it is envisioned that the channels 40 may only extend a portion thereof. Alternatively, any other insertion instrument engagement mechanism known in the art may be use such as, for example, a threaded bore, longitudinal slots, etc.

To further facilitate insertion of the implant 10, the implant 10 may also include a beveled edge along the perimeter of the superior and/or inferior surfaces 20, 22, which is devoid of an gripping structure 24 to both facilitate implant insertion and handling of the implant 10 by surgeons. More specifically, since the edges of the implant 10 are free from any gripping structure 24, the perimeter edge of the implant 10 is unlikely to become snagged by tissue during implant insertion and a surgeon is less likely to tear protective gloves while handling the implant 10 prior to and during insertion.

As previously stated, the implant 10 may include one or more radiopaque markers, especially where the implant 10 is formed from a radiolucent and/or bioresorbable material. The radiopaque marker may be embedded anywhere in the implant 10. Radiopaque markers improve visualization of the implant 10 both during and after insertion of the implant 10 into the removed disk space when the implant 10 is formed of a substantially radiolucent material. Thus, radiopaque markers indicate the position of the implant 10 with respect to the vertebral bodies and thus permit surgeons to track the progression and status of the fusion procedure through the use of X-rays or similar devices. The radiopaque marker may be made from and material known to one of ordinary skill in the art. Preferably, however, the radiopaque marker is made from barium and/or iodine.

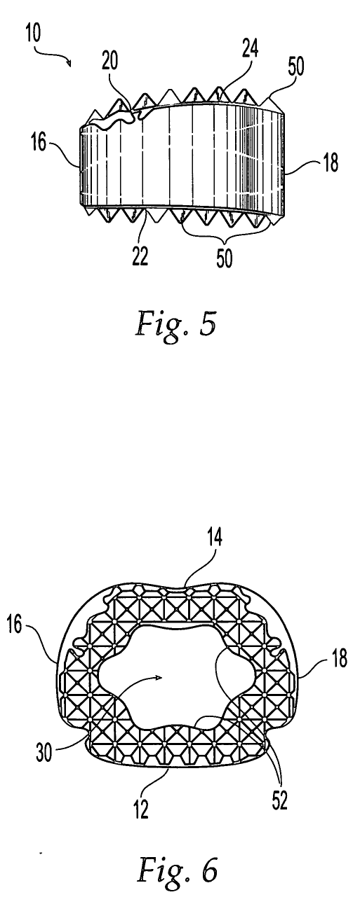

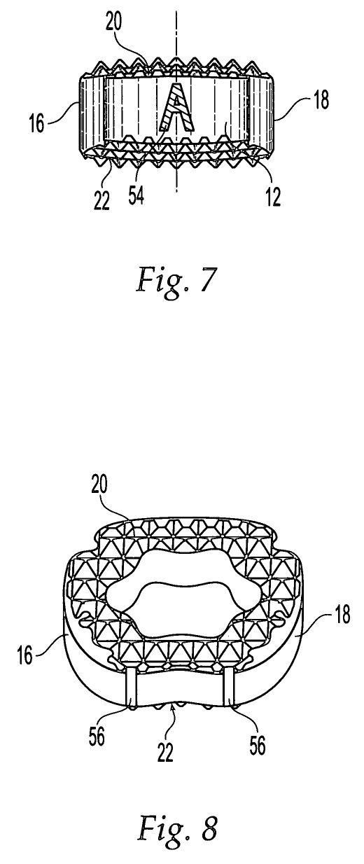

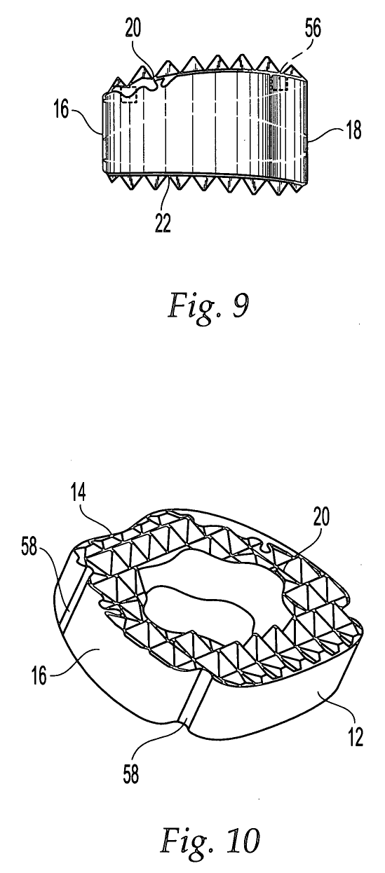

Preferably, as shown in FIG. 5, the implant 10 may include one or more teeth 50 which are filled of radiopaque material. Alternatively or in addition, as shown in FIG. 6, the outer perimeter 31 of the central bore 30 may be coated 52 with radiopaque material. Moreover, as shown in FIG. 7, one of the surfaces of the implant 10 may be stamped with a reference 54 made of radiopaque material. Preferably, the reference 54 is such that it indicates to the surgeon the proper orientation of the implant 10. For example, the anterior surface 12 of the implant 10 may be stamped with the letter “A.” As shown in FIGS. 8 and 9, the implant 10 may include one or more voids 56 which are filled with radiopaque material. Alternatively, as shown in FIG. 10, the outer perimeter of the implant 10 may include radiopaque striping 58.

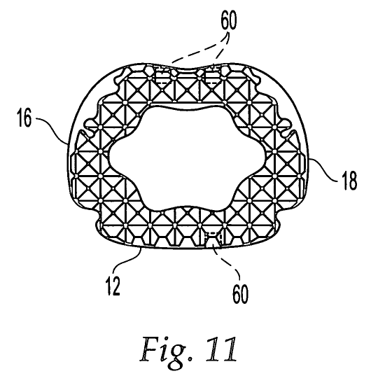

As previously stated, the implant 10 also preferably include a central bore 30 for receiving bone growth inducing substances to allow for bony in-growth and to further assist in the fusion of the vertebrae and the implant 10. Although any bone growth inducing substances known in the art may be used preferably the central bore is packed with a β-tricalcium phosphate, such as chronOS™ manufactured and sold by Synthes® Spine. ChronOS™ is manufactured from a biocompatible, radiopaque material, β-tricalcium phosphate (“β-TCP”). ChronOS™ is ideally suited for placement within the central bore 30 since it is gradually absorbed by the patient's body and replaced by new bone. Furthermore, the radiopaque natural of chronOS™ enables visualization of the implant 10. Moreover, as shown in FIG. 11, the implant 10 may contain one or more voids 60 for the insertion of bone growth inducing substances such as chronOS™.

The implant 10 is generally sized for anterior, lateral, or anterio-lateral approaches where inserting the implant around the spinal cord or spinal dural sac is not necessary as in a posterior approach. The dimensions of the implant 10 may vary depending on where in the spine the implant is inserted. Generally speaking, the vertebral bodies in the lumbar region of the spine are larger than the vertebral bodies in the thoracic region, and the vertebral bodies in the thoracic region are larger than the vertebral bodies in the cervical region of the spine. Therefore, an implant intended for the cervical region will be smaller than one for the thoracic region which will be smaller than one for the lumbar region. Likewise, an implant intended for the lower lumbar region would be larger than an implant intended for the upper lumbar region. A person of ordinary skill in the art could adapt the basic dimensions of the present invention to make them occupy the space formerly occupied by the particular removed disk which needs replacement. Thus, unless specifically specified, the dimensions of the implant are in no way intended to be limiting of the present invention. An exemplary embodiment of the implant 10 may have a depth (extending from anterior surface 12 to the posterior surface 14) ranging from 15 mm-40 mm, but preferably about 22-26 mm, and a width (extending from lateral surface 16 to lateral surface 18) ranging from 20 mm-50 mm, but preferably about 28-32 mm. In addition, in an exemplary embodiment, the height of the spinal implant 10, measured as the distance between the superior surface 20 and the inferior surface 22, when used as an intervertebral spacer, may be in the range of about 5 mm to about 25 mm. When using the spinal implant 10 as a corpectomy device, the height of the implant 10 may range from about 17 mm to about 100 mm.

The present invention has been described in connection with the preferred embodiments. These embodiments, however, are merely for example and the invention is not restricted thereto. It will be understood by one of ordinary skill in the art that other variations and modifications can easily be made within the scope of the invention as defined by the appended claims, thus it is only intended that the present invention be limited by the following claims.

What is claimed is:

1. A spinal implant for insertion between vertebrae bodies, the implant comprising an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein the central bore has a generally lobe-shaped footprint, which includes a plurality of peeks and valleys as one moves along a perimeter of the bore.

2. The implant of claim 1, wherein at least a portion of the anterior and posterior surfaces are curved and the lateral surfaces are substantially convex for mating with the curved anterior and posterior surfaces.

3. The implant of claim 2, wherein the posterior surface includes a concave recess for avoiding a foramen of the vertebral bodies when inserted.

4. The implant of claim 1, wherein the superior and inferior surfaces include a plurality of gripping structures formed thereon to facilitate engagement of the implant with the vertebrae bodies.

5. The implant of claim 4, wherein the gripping structure is a plurality of teeth.

6. The implant of claim 1, wherein the implant has a generally wedge-shaped profile extending from the anterior surface to the posterior surface.

7. The implant of claim 1, wherein one of the superior and inferior surfaces has a convexly curved surface extending substantially from the anterior surface to the posterior surface and wherein the other one of the superior and inferior surfaces has a substantially constant taper extending from the anterior surface to the posterior surface.

8. The implant of claim 7, wherein the implant has a height, the height of the implant at the posterior surface being less than the height of the implant at the anterior surface with the greatest height occurring someplace therebetween.

9. The implant of claim 7, wherein the superior and inferior surfaces both have a convexly curved surface extending from one lateral surface to the other lateral surface such that a thickness of the implant is greatest at a mid-section between the two lateral surfaces.

10. The implant of claim 1, wherein the anterior surface of the implant includes a pair of vertical channels sized and configured to engage an insertion instrument.

11. The implant of claim 10, wherein the vertical channels extend from the superior surface to the inferior surface.

12. The implant of claim 10, wherein the insertion instrument is sized and configured to engage the implant so that, once engaged, the insertion has a width substantially equal to the width of the implant.

13. The implant of claim 1, wherein the implant includes at least one radiopaque marker embedded within the implant.

14. The implant of claim 13, wherein the radiopaque marker is selected from the group consisting of:
(a) filling at least one tooth with radiopaque material;
(b) coating at least a portion of an outer surface of the central bore with radiopaque material;
(c) placing a reference marker on one of the surfaces of the implant, the reference marker being made from a radiopaque material;
(d) filling at least one void formed in a surface of the implant with a radiopaque material; and
(e) marking at least a portion of the outer perimeter of the implant with a radiopaque stripe.

15. The implant of claim 1, wherein the central bore is filled with a bone growth inducing substance, the bone growth inducing substance being chronOS™.

16. The implant of claim 1, wherein the implant includes at least one void in a surface thereof filled with chronOS™.

17. The implant of claim 1, wherein the implant is formed from a bioresorbable material.

18. The implant of claim 21, wherein the bioresorbable implant includes at least one radiopaque marker embedded within the implant.

19. The implant of claim 1, wherein the superior and inferior surfaces are convexly curved surfaces extending from one lateral surface to the other lateral surface.

20. The implant of claim 1, wherein one of the superior and inferior surfaces is curved in one plane and the other one of the superior and inferior surfaces is curved in two planes.

21. A spinal implant for insertion between vertebrae bodies, the implant comprising an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein the anterior surface of the implant includes a pair of vertical channels sized and configured to engage an insertion instrument.

22. The implant of claim 21, wherein the vertical channels extend from the superior surface to the inferior surface.

23. The implant of claim 21, wherein the insertion instrument is sized and configured to engage the implant so that, once engaged, the insertion has a width substantially equal to the width of the implant.

24. The implant of claim 21, wherein at least a portion of the anterior and posterior surfaces are curved and the lateral surfaces are substantially convex for mating with the curved anterior and posterior surfaces.

25. The implant of claim 24, wherein the posterior surface includes a concave recess for avoiding a foramen of the vertebrae bodies when inserted.

26. The implant of claim 21, wherein the superior and inferior surfaces include a plurality of gripping structures formed thereon to facilitate engagement of the implant with the vertebrae bodies.

27. The implant of claim 26, wherein the gripping structure is a plurality of teeth.

28. The implant of claim 21, wherein the implant has a generally wedge-shaped profile extending from the anterior surface to the posterior surface.

29. The implant of claim 21, wherein one of the superior and inferior surfaces has a convexly curved surface extending substantially from the anterior surface to the posterior surface and wherein the other one of the superior and inferior surfaces has a substantially constant taper extending from the anterior surface to the posterior surface.

30. The implant of claim 29, wherein the implant has a height, the height of the implant at the posterior surface being less than the height of the implant at the anterior surface with the greatest height occurring someplace therebetween.

31. The implant of claim 29, wherein the superior and inferior surfaces both have a convexly curved surface extending from one lateral surface to the other lateral surface such that a thickness of the implant is greatest at a mid-section between the two lateral surfaces.

32. The implant of claim 21, wherein the implant includes at least one radiopaque marker embedded within the implant.

33. The implant of claim 32, wherein the radiopaque marker is selected from the group consisting of:
(a) filling at least one tooth with radiopaque material;
(b) coating at least a portion of an outer surface of the central bore with radiopaque material;
(c) placing a reference marker on one of the surfaces of the implant, the reference marker being made from a radiopaque material;
(d) filling at least one void formed in a surface of the implant with a radiopaque material; and
(e) marking at least a portion of the outer perimeter of the implant with a radiopaque stripe.

34. The implant of claim 21, wherein the central bore is filled with a bone growth inducing substance, the bone growth inducing substance being chronOS™.

35. The implant of claim 21, wherein the implant includes at least one void in a surface thereof filled with chronOS™.

36. The implant of claim 21, wherein the implant is formed from a bioresorbable material.

37. The implant of claim 36, wherein the bioresorbable implant includes at least one radiopaque marker embedded within the implant.

38. The implant of claim 21, wherein the superior and inferior surfaces are convexly curved surfaces extending from one lateral surface to the other lateral surface.

39. The implant of claim 21, wherein one of the superior and inferior surfaces is curved in one plane and the other one of the superior and inferior surfaces is curved in two planes.

40. The implant of claim 21, wherein the central bore has a generally lobe-shaped footprint, which includes a plurality of peeks and valleys as one moves along a perimeter of the bore.

41. A spinal implant for insertion between vertebrae bodies, the implant comprising an anterior surface, a posterior surface, first and second lateral surfaces extending therebetween, a superior surface for engaging one of the vertebrae bodies, an inferior surface for engaging the other vertebrae body, and a central bore which extends from the superior surface to the inferior surface, wherein one of the superior and inferior surfaces has a convexly curved surface extending substantially from the anterior surface to the posterior surface and wherein the other one of the superior and inferior surfaces has a substantially constant taper extending from the anterior surface to the posterior surface, the superior and inferior surfaces both having a convexly curved surface extending from one lateral surface to the other lateral surface.

42. The implant of claim 41, wherein at least a portion of the anterior and posterior surfaces are curved and the lateral surfaces are substantially convex for mating with the curved anterior and posterior surfaces.

43. The implant of claim 42, wherein the posterior surface includes a concave recess for avoiding a foramen of the vertebral bodies when inserted.

44. The implant of claim 41, wherein the superior and inferior surfaces include a plurality of gripping structures formed thereon to facilitate engagement of the implant with the vertebrae bodies.

45. The implant of claim 44, wherein the gripping structure is a plurality of teeth.

46. The implant of claim 41, wherein the anterior surface of the implant includes a pair of vertical channels sized and configured to engage an insertion instrument.

47. The implant of claim 46, wherein the vertical channels extend from the superior surface to the inferior surface.

48. The implant of claim 46, wherein the insertion instrument is sized and configured to engage the implant so that, once engaged, the insertion has a width substantially equal to the width of the implant.

49. The implant of claim 41, wherein the implant includes at least one radiopaque marker embedded within the implant.

50. The implant of claim 49, wherein the radiopaque marker is selected from the group consisting of:
(a) filling at least one tooth with radiopaque material;
(b) coating at least a portion of an outer surface of the central bore with radiopaque material;
(c) placing a reference marker on one of the surfaces of the implant, the reference marker being made from a radiopaque material;
(d) filling at least one void formed in a surface of the implant with a radiopaque material; and
(e) marking at least a portion of the outer perimeter of the implant with a radiopaque stripe.

51. The implant of claim 41, wherein the central bore is filled with a bone growth inducing substance, the bone growth inducing substance being chronOS™.

52. The implant of claim 41, wherein the implant includes at least one void in a surface thereof filled with chronOS™.

53. The implant of claim 41, wherein the implant is formed from a bioresorbable material.

54. The implant of claim 53, wherein the bioresorbable implant includes at least one radiopaque marker embedded within the implant.

55. The implant of claim 41, wherein the central bore has a generally lobe-shaped footprint, which includes a plurality of peeks and valleys as one moves along a perimeter of the bore.
